(19) United States
(12) Patent Application Publication
(10) Pub. No.: US 2005/0287125 A1
(43) Pub. Date: Dec. 29, 2005

(54) Cell delivery fluid for prevention of cell settling in delivery system

(75) Inventors: Mary M. Morris, Mounds View, MN (US); Matthew A. Bergan, Forest Lake, MN (US); Brian C.A. Fernandes, Roseville, MN (US); Kenneth C. Gardeski, Plymouth, MN (US); Stanten C. Spear, Arden Hills, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 11/200,345

(22) Filed: Aug. 8, 2005

Related U.S. Application Data
(63) Continuation-in-part of application No. 10/442,525, filed on May 21, 2003.
(60) Provisional application No. 60/382,764, filed on May 22, 2002.

(51) Int. Cl. A61K 45/00; A01N 1/02

(52) U.S. Cl. 424/93.7; 435/2

(57) ABSTRACT

A method involves selecting a type of cell for implantation into a mammal and identifying the specific gravity or density of the cell type. Then, a carrier liquid is selected which has a specific gravity or density within a range of specific gravities or densities which approximately matches the specific gravity or density of the selected cell type. A liquid for delivery of growth cells into tissue of a mammal is also provided in which the density of a carrier fluid component is matched to the density of the cells being consistently delivered by the carrier fluid.

### CROSS-REFERENCE TO RELATED APPLICATIONS

The present application is a continuation-in-part of U.S. application Ser. No. 10/442,525, filed May 21, 2003, which claims priority to U.S. Provisional Application No. 60/382,764, filed May 22, 2002, the entire disclosure of which are incorporated herein by reference.

### FIELD OF THE INVENTION

The invention relates to a medical device-related cell delivery carrier medium, and methods of delivering cells for generating tissue growth.

### BACKGROUND OF THE INVENTION

Previously, there has been interest in the delivery of cells to locations within mammalian bodies to effect new growth of tissue. This technology is designed to promote growth of new tissue from implanted cells, often originating from the same mammal receiving the cells, and designed to generate new tissue in the region of implantation. Various types of tissue may be implanted, including for example, bone, cartilage, muscle and other types.

Cardiac tissue has also been the subject of cell delivery efforts in order to repair cardiac walls and other regions severely damaged by myocardial infarctions or congestive heart failure. One example of such use includes skeletal muscle-derived myoblasts or stem cells delivered surgically into the myocardium of the patient to regenerate damaged tissue, promote revascularization and angiogenesis. Desired volume concentrations of cells per delivery vary according to indications, but it is not uncommon to have tens to hundreds of millions of cells intended to be delivered to one or more sites.

### SUMMARY OF THE INVENTION

The invention involves the recognition of the problem of cell settling during the delivery of cells to designated sites, thus preventing the intended concentrations of cells from being delivered. In order to overcome this problem, and subsequent delivery inaccuracies, a method is provided of preparing a cell-carrier liquid suspension for implantation into a mammal which prevents cell settling. The method involves selecting a type of cell for implantation into a mammal and identifying the specific gravity of the cell type. Then a carrier liquid is selected having a specific gravity within a range of specific gravities. This enables an approximate match of the specific gravity of the carrier liquid with the selected cell type such that the cells do not tend to float or settle, but stay in suspension to allow delivery of an acceptable number of viable cells at one or more delivery sites in the mammal. It is further desired to ensure that the carrier liquid has an appropriate osmolality and pH to ensure an acceptable viability ratio of the cells at the delivery destination. By mixing the carrier liquid and the cells into a proper liquid suspension, it is possible to achieve a more accurate and consistent delivery of the intended amount of specific cells at a specific site—either percutaneously or surgically.

A cell-carrier liquid suspension for delivery into tissue of a mammal is also provided in which the density of a carrier fluid component is matched to the density of the cells of interest. Accordingly, the cell-carrier liquid suspension provides a tissue forming liquid suspension with greater efficacy in treating defective tissue.

### BRIEF DESCRIPTION OF THE DRAWINGS

FIG. 1 depicts a graph of top and bottom cell count ratios of fibroblasts suspended in cell suspension medium.

FIG. 2 depicts a graph of a ratio of top and bottom total cell counts of fibroblasts suspended in ISOVUE® cell density matched media according to the teachings of the present invention.

FIG. 3 depicts a graph of myoblast cell settling in cell density matched media according to the teachings of the present invention.

FIG. 4 is a table of catheter values of myoblast cell settling in cell density matched media according to the teachings of the present invention.

FIG. 5 consists of images taken during cell settling in cell density matched media according to the teachings of the present invention.

FIG. 6 is a table of delivery dynamics for myoblast and fibroblast cell delivery through catheters myoblast cell settling in cell density matched media according to the teachings of the present invention.

FIGS. 7A-H are SEM photographs of the inner lumens of catheters after the delivery of cells in cell density matched media according to the teachings of the present invention.

FIG. 8 depicts maintenance of cell suspension over time in cell density matched media according to the teachings of the present invention.

FIG. 9 depicts maintenance of cell suspension over time in cell density matched media including albumin according to the teachings of the present invention.

### DETAILED DESCRIPTION OF THE INVENTION

The present invention provides methods and compositions for accurate, predictable delivery of cells in suspension to a targeted delivery site within a mammal. The methods and compositions contemplate delivery of a more accurate and precise amount of cells and suspension liquid in order to grow new tissue at the target locations.

Cells which may be delivered to sites within a mammal according to the teachings of the present invention include, but are not limited to, mature myogenic cells (e.g., skeletal myocytes, cardiomyocytes, purkinje cells, fibroblasts), progenitor myogenic cells (such as myoblasts), mature non-myogenic cells (such as endothelial and epithelial cells), hematopoietic cells (monocytes, macrophages, fibroblasts, alpha islet cells, beta islet cells, cord blood cells, erythrocytes, platelets, etc.) or stem cells (pluripotent stem cells, mesenchymal stem cells, endodermal stem cells, ectodermal stem cells, whether adult or embryonic, or whether autologous, allogenic, or xenogenic). More particularly, cells which may be delivered according to this invention further include islet cells, hepatocytes, chondrocytes, osteoblasts, neuronal cells, glial cells, smooth muscle cells, endothelial cells, skeletal myoblasts, nucleus pulposus cells, and epithelial cells. Any of the mentioned cell types can be genetically engineered to contain DNA or RNA introduced into the cell by recombinant techniques. The DNA or RNA introduced into the cell may include, but are not limited to, new genes, promoters, interfering RNAs, and the like. Accordingly, tissue may be formed resulting in new growth of cartilage, bone, skin, epithelial layers, new organs, central nervous system tissue, and muscle-including that tissue appropriate for re-generation of certain cardiac function. It is recognized that the methods and compositions, along with the essential aspects of the invention, may allow delivery of numerous types of cells whether listed above, by example, or not, in a much improved manner and with increased efficacy. It is recognized that any of the above mentioned cells used in conjunction with the cell density matched carrier fluids may be labeled or tagged with radio-isotope labels, fluorescently tagged, enzymatically tagged, or further used in combination with specific cell protein antibody markers.

The challenge of accurate cell delivery (and accompanying nutrients) has been addressed by use of a gel matrix (or similar structures) as a carrier medium, such as shown in U.S. Pat. Nos. 6,171,610 B1; 6,129,761; 5,667,778. Typically, the cells are placed in suspension in hydrogels in order to stabilize the cells at the delivery site. However, problems arise from use of the gels due to the relatively high pressure required to inject the high viscosity gels via a catheter, which is generally considered a less invasive approach than a direct surgical path. Moreover, gels utilize material having relatively longer molecules and higher viscosities that causes increased shear stress during delivery resulting in more cell damage. Alternatively, gels delivered surgically or endoscopically must also share the risk factors and inefficiencies attendant to those procedures.

Another type of cell delivery medium is that shown in U.S. Pat. No. 5,543,316, which describes an injectable composition comprising myoblasts and an injectable grade medium having certain components designed for maintaining viability of the myoblasts for extended periods of time. The osmolality of the medium is preferably from about 320 mOsm/kg to about 550 mOsm/kg (e.g, more preferably selected from the osmolality of about 250 mOsm/kg, about 300 mOsm/kg, about 350 mOsm/kg, 400 mOsm/kg, about 450 mOsm/kg, about 500 mOsm/kg, about 550 mOsm/kg, about 600 mOsm/kg, and the like). The term osmolality generally refers to the concentration of an osmotic solution especially when measured in osmols (osm/kg) or milliosmols (mOsm/kg) per 1000 grams of solvent. This technique, combined with attempted delivery of very high concentrations of cells, represents another method of overcoming the challenges of effective cell delivery therapy.

The above reference is one example of the past misunderstanding regarding the cause of cell delivery inaccuracies. In the past it has been assumed that cell death was the cause of cell delivery problems, such as that caused by shear stress induced by a combination of time, pressure, diameter of delivery vehicle lumen, and the size of the cells being delivered. What was not realized was that cell settling in liquid solutions was an important cause of delivery inaccuracies.

What has not been realized or identified is a simple solution to the problem of inconsistent concentrations of cells being delivered in a liquid medium. Applicants have recognized this problem and identified a cell delivery medium having characteristics designed to overcome this obstacle to effective therapy. The cell delivery medium is density matched with the cells it is delivering. The cell delivery medium may be deliverable at pressures of less than about 1500 psi. More specifically, when the cell delivery medium of the present invention is delivered by hand (i.e., via. a syringe or other mechanical device, wherein force is directly applied by a user to create a pressure gradient), it is preferred that the cell delivery medium has a low enough viscosity to be deliverable at pressures of less than about 200 psi (including but not limited to such pressures as about equal to or less than 180 psi, 160 psi, 140 psi, 120 psi, 100 psi, 80 psi, 60 psi or 40 psi, and the like). The abbreviation psi as used herein means one pound per square inch. When the cell delivery medium is delivered by a non-hand operated device (i.e., a pump or other device (mechanical, electrical, etc.) that creates a pressure gradient without force directly applied by a user) it is preferred that the cell delivery medium has a low enough viscosity to be deliverable at pressures of less than about 1500 psi.

One skilled in the art recognizes that the internal diameter (I.D.) of the delivery tube affects the fluid dynamics of delivered solutions. For example, in a 0.012 inch inner diameter (0.30480 millimeter), 60 inch (1.524 meter) length catheter it was possible to readily deliver a 1 centipoise fluid but not a 5 centipoise fluid at the pressures used. In a similar example, in a 0.017 inch inner diameter (0.43180 millimeters), 12 inch length (30.480 cenitmeter) catheter it was possible to readily deliver fluids up to and including 50 centipoise. These characteristics will optimize cell viability, ease of physician delivery, and patient comfort and recovery.

It is recognized then that various internal diameters of catheters can be used with selected cell density solutions (including, but not limited to, 0.017 in. (0.43180 millimeter), 0.016 in. (0.40640 millimeter), 0.014 in. (0.35560 millimeter), 0.0135 in. (0.34290 millimeter), 0.0012 in. (0.30480 millimeter), 0.009 in. (0.22860 millimeter) and the like). The abbreviation in. indicates a measurement in inches. Similarly, because of the high survivability rate demonstrated for cells in these solutions, much higher shear rates can be used than previously believed possible, including, but not limited to rates equal to or greater than 1000 1/sec, 2000 1/sec, 3000 1/sec, 4000 1/sec, 5000 1/sec, 6000 1/sec, 7000 1/sec, 8000 1/sec. One feature of the described cell delivery fluids is that they permit cells to survive much higher shear stress in catheters (including but not limited to equal or greater than 1 N/m², 2 N/m², 3 N/m², 4 N/m², 5 N/m², 6 N/m² and the like). The abbreviation N/m² indicates a measurement of force and means Newtons per meter squared. One skilled in the art would recognize that the survivability of cells is proportional to the shear stress in the catheter and the length of time it experiences the effective shear forces. It is recognized that the effective time that a cell experiences an effective shear stress in the catheter may be as short as about 10 msec to upward of 5000 msec (including ranges of less then 4000 msec, less then 3000 msec, less then 2000 msec, less then 1000 msec.) The abbreviation msec is a unit of time that is one millisecond (one thousandth of a second). Therefore, ideal survival rates for cells may be optimized by effectively matching the delivery requirements, the shear stress, and the delivery time.

It is also recognized that higher viscosities may be possible with cell delivery devices via a more direct surgical approach in delivery devices of relatively shorter length and possibly of a larger lumen size, and still enjoy the benefits of this invention. However, a cell carrier liquid medium must be density (or specific gravity) matched with the cells it is transporting for optimal results. However, the present invention may use less than optimally matched cell density carriers where the use of these carriers with the delivered cells improves at least one measurable fluid dynamic in the catheter or at least one measure of effective delivery. Consistent with the foregoing matching of density carriers is that the cell density solution may be within about +/−10%, within about 5%, within about 2%, within about 1%, within about 0.1%, or within about 0.01% of any given cell density. Known examples of media which may be appropriate, with proper formulation, include ISOVUE® brand image enhancing media (sold by Bracco Diagnostics), perfluorooctyl bromide (Perflubron™), known under the Oxygent™ brand name (sold by Alliance Pharmaceuticals), dextran solutions, such as Dextran™ 40 I.P, Microspan-40™ in normal saline, and MICROSPAN40™ in 5% dextrose (Claris Life Sciences).

In another embodiment of the present invention one or more molecular components may be added to the solution. These molecular components generally comprise protein(s) and/or non-protein(s). Any individual or combination of protein(s) and/or non-protein(s) may be added to the solution. These proteins include, but are not limited to, cytokines, chemokines, and growth factors (i.e., those growth factors involved in cell proliferation, migration, differentiation, cell signaling, etc.). Various examples of growth factors include platelet derived growth factors (PDGF), vascular endothelial growth factors (VEGF) and its family of proteins, fibroblast growth factor (FGF), epidermal growth factor (EGF), insulin-like growth factor (IGF), transforming growth factor (TGF), neurotropic growth factor (NGF), etc. These proteins may also include extracellular matrices. These matrices may include structural matrices (i.e, collagens (types I, II, III, IV, etc.), and elastin, etc.), adhesion and/or migration matrices (i.e., fibronectin, laminin, tenascin, entactin, fibrinogen, fibrin, etc.) and/or proteoglycans (i.e. heparin and/or heparin sulfate, dermatan sulfate, keratan sulfate, chondroitin sulfate, etc.). Additional types of proteins included herein are enzymes and enzyme inhibitors. Once example of an enzyme inhibitor includes tissue inhibitors of matrix metalloproteinases (TIMPS). Other proteins included herein are antibodies, peptides (i.e., those containing the RGD sequence) protein derivaties (i.e, gelatin) and albumin.

Non-proteins may also be included either alone or in combination with the type(s) of proteins disclosed herein. Non-limiting examples of non-proteins contemplated herein include polysaccharides (i.e, hyaluronan, hyaluronic acid, dextrans and/or modified dextrans, etc.) and prostaglandins.

Also contemplated herein are various protein mixtures, wherein one or more proteins is mixed and/or combined with at least one other protein (i.e., protein-protein combinations, including those combinations involving serum-derived proteins and/or serum itself). The proteins contemplated herein may be natural or recombinant proteins.

At the present time, it is applicants' belief that the addition of a molecular component (i.e., a protein and/or a non-protein) to the cell medium carrier will not substantially alter the viscosity of the cell medium carrier. The term “substantially alter” as used herein means that the addition of a molecular component to a cell medium carrier will not substantially alter the viscosity of the cell medium carrier by more than +/−1 centpoise (compared to a cell medium carrier without a molecular component). The measure of viscosity is commonly measured in centipoise (cP). That is to say, the addition of a protein and/or a non-protein to the cell medium carrier will not substantially affect the cell settling rate in the cell medium carrier. Rather, as described herein, the present invention includes a cell delivery carrier medium (i.e., a liquid suspension) that includes select cells and a carrier liquid where the density of the cells substantially matches the density of the carrier liquid, wherein the cells remain substantially displaced in the carrier liquid and a method of delivering the same.

An exemplary protein is human serum albumin (HSA), typically a crystallizable albumin or a mixture of albumins that normally comprise more than half of the protein in the blood serum. HSA also serves to maintain the osmotic pressure of the blood. HSA generally enhances the expression of various types of cell growth factors and, in some cases, up-regulates cell growth receptors.

In another embodiment of the present invention, the cell density matched solutions may also serve as image enhancing agents. Image enhancing agents can be effectively used for imaging the delivered fluid and for balancing cell density. Such reagents include iodine based solutions (such as iopamidol, sold as ISOVUE® by Bracco Diagnostics), gadodiamide (OMNISCAN® sold by Claris Life Sciences), and InFeD® (iron dextran manufactured by Schein Pharmaceutical).

In yet another embodiment of the present invention, the cell density matched solutions may also be labeled or tagged to aid in its detection. A number of the mentioned reagents are amenable to being synthesized with radioactive elements or radioactively tagged (e.g, radio C¹⁴ or H³ labeled glucose solutions, radioactive ISOVUE®, radio active iodine (I¹²⁵) regents, and the like). Image enhancing agents can be effectively used for imaging the delivered fluid and for balancing cell density. Such reagents include iodine based solutions (such as iopamidol, sold as ISOVUE® by Bracco Diagnostics), gadodiamide (OMNISCAN® sold by Claris Life Sciences), and InFeD® (iron dextran manufactured by Schein Pharmaceutical).

Methods for determining cell density are well known in the art. Listed below in Table 1 are some published density values for the cell types given:

TABLE 1
CELL SPECIFIC GRAVITIES

| | |
|---|---|
| red blood cells: | 1.10 |
| stem cells (CD34 cells): | 1.065 |
| platelets: | 1.063 |
| monocytes: | 1.068 |
| lymphocytes: | 1.077 |
| hepatocytes | 1.07-1.10 |
| granulocytes | 1.08-1.09 |

The values in Table 1 are given to be exemplary and not limiting, and can be experimentally determined for the relevant cell population. Also, the term specific gravity as used herein means the ratio of the density of a substance (ie, the cell) to the density of some other substance (i.e. pure water) taken as a standard when both densities are obtained by weighing in air. For example, PERCOLL® (a product of Pharmacia) is a well referenced medium for density gradient centrifugation of cells. PERCOLL® will form self-generated gradients during centrifugation so that cells mixed with PERCOLL® prior to centrifugation will band isopycnically as the gradient is formed in situ. PERCOLL® can be used with density marker beads (Sigma product # DMB-10) which can be used to identify density levels within a PERCOLL® gradient. PERCOLL® is a synthetic, colloidal solution of polyvinylpyrrolidone coated silica, specifically designed for sedimentation centrifugation.

The number of cells delivered suspended in the described liquid carriers may vary widely in the actual effective cell concentration. The cell concentration may vary from about 1×10⁹ cells per milliliter to about 1×10⁶ cells per milliliter (ml) (including from about 1×10⁹ cells/ml, about 5×10⁸ cells/ml, about 1×10⁸ cells/ml, about 5×10⁷ cells/ml, about 1×10⁷ cells/ml, about 5×10⁶ cells/ml, about 1×10⁶ cells/ml, and the like depending on cell size). Choice of the delivered concentration of cells along with the number of cells is one criteria matched in selecting the appropriate delivery carrier for the delivered cells and medium to the target site.

One of several goals of the carrier medium of this invention is to mitigate or prevent undesired settling of the cells placed in the carrier medium. This is done in order to achieve a known, consistent (and preferably very high) cell delivery concentration ratio, i.e., delivered cells as compared with available cells intended to be delivered by the physician to a specific site should be close to the value of 1:1. It is a similar goal to ensure that an acceptable viability ratio (preferably also near 1:1) is achieved by which a high percentage of delivered cells are functional and replicate at well accepted levels. Methods and compositions which achieve this goal provide a significant improvement over the known methods for delivery of high number of viable cells to diseased or degenerated tissues. Another goal of the present invention is to eliminate the need to use mechanical mixing devices and/or mechanical mixing methods once the cells have been combined with the carrier medium prior to administration. More specifically, because once the cells are combined with the carrier liquid the cells are held in suspension, there is no need to use a mechanical mixing device and/or a mechanical mixing method to maintain the cells in suspension.

Applicants realized through investigation that cell loss, rather than cell death, was possibly the critical issue in catheter-based cell delivery. Following that realization, certain experiments validated cell loss as being caused by cell settling rather than adhesion to the delivery structure. Applicants also realized that through investigation the issue of cell settling is a critical component to any cell delivery method whether through use of pumps or catheters. Catheter applications would include use with cardiac delivery catheters; epicardial, endocardial, pericardial, intralumenal (e.g., intracoronary, intravenous) and the like. Use of cell density balanced solutions also would be appropriate in other non-cardiac delivery methodologies, such as neurovascular, peripheral vascular, and the like, and more generally to syringe delivery of cells.

### EXAMPLES

### Example I

Fibroblast cells were stored in 50 mL centrifuge tubes over a period of 100 minutes, both on ice and at room temperature (RT). Samples were removed by pipette from the top and bottom of both the ice and RT suspensions every 20 minutes. No mixing was done for the first 60 minutes. At the 80 minute time point, gentle mixing (hand swirling) was done immediately before sampling. At the 100 minute time point, hard mixing (vigorous hand swirling) was done immediately before sampling

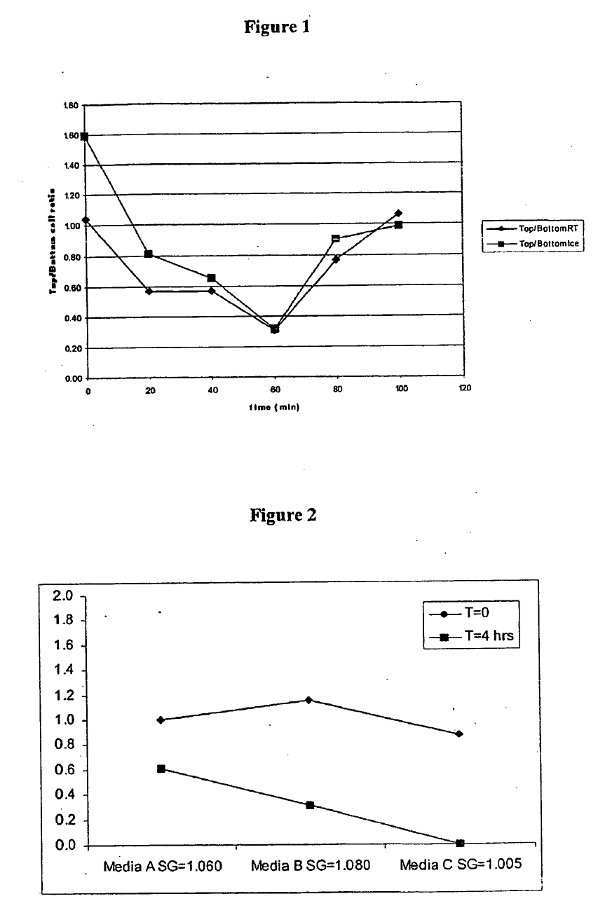

FIG. 1 illustrates the top/bottom cell count ratios as the results of this experiment. The stratification of a static fibroblast suspension, whether kept at room temperature or on ice, is clearly demonstrated. By 60 minutes, the cell concentration taken from the top of the suspension was only 30% of that taken from the bottom. But after a gentle mix (the 80 minute time point), suspension equilibrium was clearly restored.

The results shown in Example I demonstrate that fibroblast suspensions do not maintain their initial concentration when allowed to sit over time. The results suggest that settling of the suspensions is occurring, but that even gentle mixing brings these suspensions back into equilibrium. This finding has potential impact on delivery device, delivery medium, and overall delivery system design, as it will be critical to assure that the appropriate concentration of therapeutic cells can be delivered through the catheter repeatedly and reliably. However, recognizing that rapid settling may occur, then constant agitation of a delivery vehicle and injectate medium may be necessary to prevent such phenomenon. But as a practical matter such agitation is not desirable by the physician. Consequently, Applicants identified a new solution to achieve a matched density of the carrier medium with the cells being delivered by that medium.

Prevention of human fibroblast settling was investigated using isotonic diluted solutions of ISOVUE® brand image enhancing media to increase the specific gravity of the cell media above normal saline

### Example II

A cell delivery medium was density matched with cells as a dilution of ISOVUE®-300 image enhancing media (sold by Bracco Diagnostics) and human dermal fibroblasts. ISOVUE® is a non-ionic image enhancing media with the active agent of iopamidol. The package insert for ISOVUE®-300 lists the concentration as 300 mg/mL (61%), osmolality of 616 mOsm/kg water, viscosity at 20° C. as 8.8 cP, and specific gravity of 1.339. ISOVUE®-300 was then diluted 1:2 v/v (1 part ISOVUE® to 1 part deionized water). The 1:2 diluted ISOVUE® osmolality is about 300 mOsm/kg, and the calculated specific gravity is 1.170. The fibroblasts suspended in Hanks Balanced Salt Solution (HBSS) were then diluted with the diluted Isovue media to achieve a specific gravity of 1.060. Since the osmolality of both HBSS and diluted ISOVUE® media is about 300 mOsm/kg, the dilution does not change the osmolality of the diluted cell suspensions.

The specific gravities of the solutions tested were 1.060 (Media A), 1.080 (Media B), and 1.005 (control-Media C). The cell concentrations on the top and bottom of the three solutions were counted before and after 4 hours of settling time. As shown in FIG. 2, both the high density solutions significantly slowed the fibroblasts settling compared to the control. A comparison of top layers over time can also be made. After 4 hours, the control had zero cells in the top layer, and the diluted ISOVUE® solutions had 105 & 57% of the initial cell count in the top layer. After 4 hours, the bottom layer for all the solutions contained more cells than counted initially.

Trypan Blue™ cell counts (using Trypan Blue™ solution from Sigma Chemical) were performed at the two time points (0 & 4 hours). The number of stained (dead) cells were not significantly different for the diluted ISOVUE® solutions compared to the saline control. Diluted ISOVUE® media did not appear to significantly rupture cell membranes after 4 hours of contact. The cell proliferation assay indicated the fibroblasts proliferated after exposure to Isovue as well as with the saline control.

### Example III

This experiment was very similar to that performed in Example II, except that myoblasts were used rather than fibroblasts. Prevention of myoblast settling was investigated using isotonic diluted solutions of ISOVUE® image enhancing media to increase the specific gravity of the cell media above normal saline. The specific gravities of the solutions tested were 1.060 (Media A), 1.080 (Media B), and 1.005 (control-Media C). The cell concentrations on the top and bottom of the three solutions were counted before and after 4 hours of settling time.

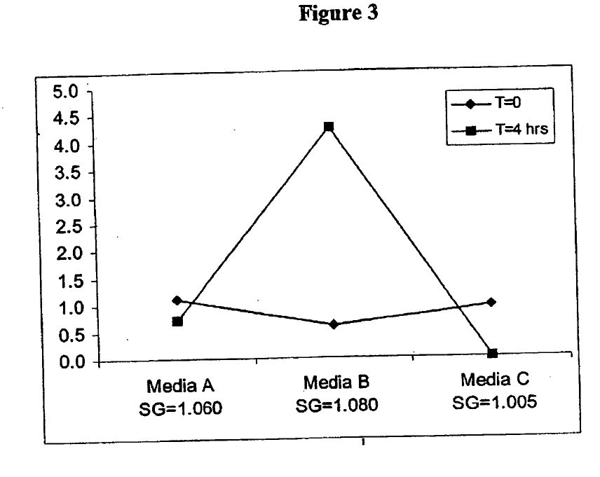

As shown in FIG. 3, both the high-density solutions significantly slowed the myoblasts settling compared to the HBSS control. After 4 hours, the control had zero cells in the top layer, and the diluted ISOVUE® solutions had 75% (Media A) and 85% (Media B) of the initial cell count in the top layer. The centrifuge tubes were gently mixed by hand swirling prior to the proliferation assay. These cell counts, after gentle mixing, indicate 24-40% of the cells were lost after four hours due to adherence to the vessel wall or each other (clumping). Cell settling was a more significant issue than adherence as the control had a 29-fold increase of cells on the bottom of the tube after four hours of settling. The density of the myoblasts is approximately 1.06 g/mL. The number of Trypan Blue™ stained (dead) cells were very few and not significantly different for the three solutions. Diluted ISOVUE® media does not appear to significantly rupture the myoblast cell membranes after 4 hours of contact. The cell proliferation assay indicates the myoblasts proliferated as well after exposure to ISOVUE® as prior to exposure.

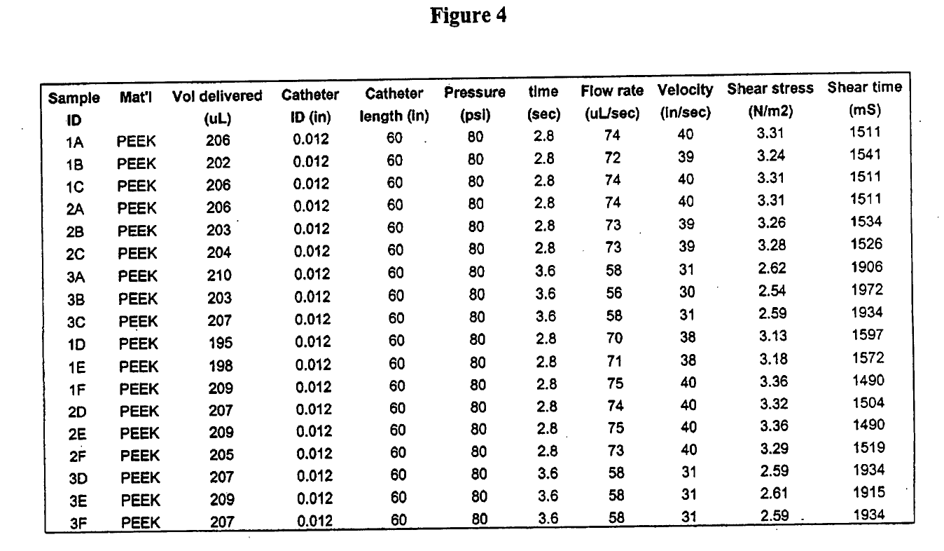

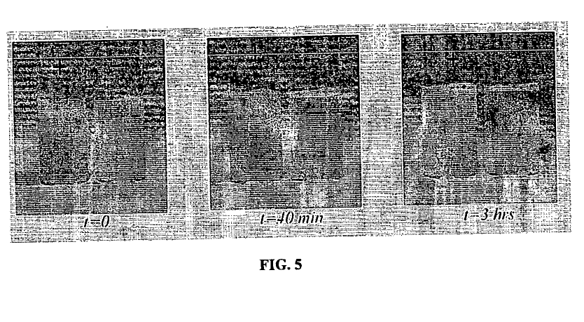

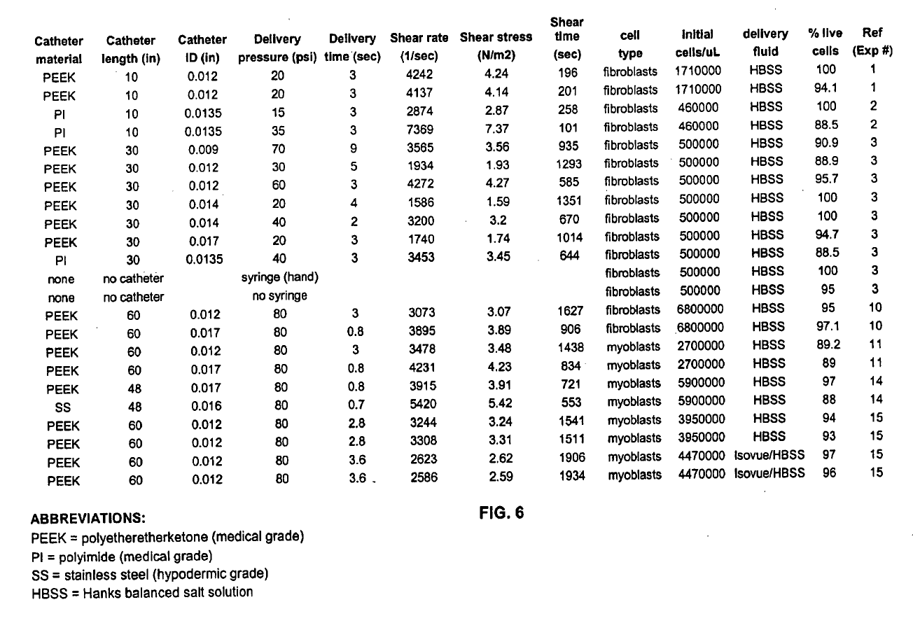

Applicants' previous experiments demonstrated that by matching the density and osmolality of a carrier fluid to that of the cells being delivered, then settling of cells can be minimized. Applicants successfully performed experiments which verified that viable, proliferative myoblasts can be delivered consistently through a wide range of catheter materials. Catheter materials may include various polymers, including but not limited to, poly etheretherketone (PEEK), polyimide (PI—medical grade), polyurethanes, polyamides, silicones, polyethylenes, polyurethane blends, polyether block amides (e.g., PEBAX®), and the like, or including various metal materials, including but not limited to stainless steel (SS), titanium alloys, nickel titanium alloys (e.g. Nitinol), chromium alloys (MP35N, ELGILOY®, Phynox, etc.), cobalt alloys, and the like. More preferably the catheter materials are chosen from the group of poly etheretherketone (PEEK), polyimide (PI), and stainless steel (SS). One feature studied when cells were delivered through catheters (after proper mixing in the cell density solution) was the behavior of myoblasts and fibroblasts in the various vessels used to hold, transport, and inject cells over the expected implant period. Accordingly, further experiments included combinations of density matched and non-density matched delivery media in catheters of lengths, lumen sizes, and materials representative of those suitable for transvenous cell delivery. As shown in FIGS. 4 and 6, and used in the investigations of Examples IV and VI below, some of the media demonstrated relatively high shear time, which was previously believed to be a key indicator of adhesion and cell delivery inaccuracies.

Example IV evaluates the two technologies of cell delivery and prevention of cell settling performed simultaneously by delivering myoblasts through catheters using cell settling prevention media. The investigation focuses on the variation of parameters and their effect on cell survival. The design parameters of interest include pressure, flow rate, catheter diameter, catheter length, and cell concentration. Concentrations and survival rates of cells delivered from the settling prevention media are measured and compared to those of cells delivered from HBSS. Cells are allowed to settle for 40 minutes in an effort to determine whether cells suspended in settling prevention media can be delivered without the need for mixing.

### Example IV

Three separate delivery solutions were prepared. The cell concentration was held constant by using the ratio of 2 mL cell suspension/i mL carrier solution for a total of 3 mL added to each of three delivery syringes. The cell suspension was the same in all three solutions. The makeup of each of the three carrier solutions was as follows:
Solutions 1 and 2: Hanks balanced salt solution as used in previous experiments
Solution 3: ISOVUE® 370/deionized (DI) water mix, adjusted for a specific gravity of 1.060 and an osmolality of 300 mOsm/kg

Solution 3 was prepared similarly to the cell settling prevention media of previous experiments, with the exception that ISOVUE® 370 image enhancing media was used in place of ISOVUE® 300. ISOVUE® 370 is simply a more concentrated iopamidol solution than ISOVUE® 300, and it was found that an additional dilution with DI H₂O (3.8 mL of DI H₂O for every 16.2 mL of Isovue 370) brought the properties of ISOVUE® 370 media equivalent to those of ISOVUE® 300 media. Dilutions then continued as per the above referenced method in Example II.

Cells and carrier solutions were mixed in 50 mL centrifuge tubes, labeled Solutions 1, 2, and 3, and used as described below. Catheter assemblies 152.4 cm (60 inches) long were built from 0.012 in ID PEEK tubing. Myoblasts were stored in 50 mL centrifuge tubes throughout the experiment and were never frozen before use. Cells were delivered through catheter assemblies by use of the EFD Model 1500XL fluid delivery system. For each experiment, the following data were collected:
Hemocytometer counts and Trypan Blue™ viability staining, on myoblasts from each of the three initial 50 mL centrifuge tubes;
Hemocytometer counts, and Trypan Blue™ viability staining, on myoblasts from each solution immediately after (t=0 minutes) the delivery through their respective catheters; and
Hemocytometer counts and Trypan Blue™ viability staining, on myoblasts from each solution immediately after (t=40 minutes) the delivery through their respective catheters.

Tables 2, 3, and 4 below show the mixing protocols for each solution. Briefly, Solution 1 is the HBSS/cells solution, mixed before t=O but not before t=40; Solution 2 is the HBSS/cells solution, mixed before both t=0 and t=40; and Solution 3 is the ISOVUE®/cells solution, mixed before t=0 but not before t=40.

TABLE 2

| | | t = 0 | t = 40 | |
|---|---|---|---|---|
| | | delivered cell | delivered cell | % of |
| Solution | | concentration | concentration | viable |
| #1 | Delivery Solution | (average) | (average) | cells |
| 1A | HBSS, no mix at 40 | 4.30 M | | 95% |
| 1B | HBSS, no mix at 40 | 3.80 M | | 94% |
| 1C | HBSS, no mix at 40 | 4.30 M | | 94% |
| 1D | HBSS, no mix at 40 | | 1.93 M | 92% |
| 1E | HBSS, no mix at 40 | | 1.43 M | 97% |
| 1F | HBSS, no mix at 40 | | 1.33 M | 95% |

TABLE 3

| | | t = 0 | t = 40 | |
|---|---|---|---|---|
| | | delivered cell | delivered cell | % of |
| Solution | | concentration | concentration | viable |
| #2 | Delivery Solution | (average) | (average) | cells |
| 2A | HBSS, mix at 40 | 4.20 M | | 93% |
| 2B | HBSS, mix at 40 | 3.83 M | | 93% |
| 2C | HBSS, mix at 40 | 3.83 M | | 93% |
| 2D | HBSS, mix at 40 | | 4.10 M | 91% |
| 2E | HBSS, mix at 40 | | 3.98 M | 91% |
| 2F | HBSS, mix at 40 | | 3.93 M | 88% |

TABLE 4

| | | t = 0 | t = 40 | |
|---|---|---|---|---|
| | | delivered cell | delivered cell | % of |
| Solution | | concentration | concentration | viable |
| #3 | Delivery Solution | (average) | (average) | cells |
| 3A | ISOVUE ®/HBSS, | 4.18 M | | 97% |
| | no mix at 40 | | | |
| 3B | ISOVUE ®/HBSS, | 4.03 M | | 96% |
| | no mix at 40 | | | |
| 3C | ISOVUE ®/HBSS, | 4.25 M | | 96% |
| | no mix at 40 | | | |
| 3D | ISOVUE ®/HBSS, | | 3.75 M | 96% |
| | no mix at 40 | | | |
| 3E | ISOVUE ®/HBSS, | | 3.93 M | 93% |
| | no mix at 40 | | | |
| 3F | ISOVUE ®/HBSS, | | 4.00 M | 94% |
| | no mix at 40 | | | |

Canine skeletal myoblasts were cultured until sufficient cells were available. The myoblasts were dissociated, rinsed, and re-suspended in HBSS into a 50 mL centrifuge tube containing the appropriate carrier solution. In this experiment Applicants were able to deliver a minimum of 1 million cells/mL into the catheters.

Cells from all samples described in the preceding sections were manually counted in duplicate using a hemocytometer. Table 5 shows the cell concentrations, with units in cells/mL, and also various cell concentration ratios.

TABLE 5

| | | | t = 0 delivered | t = 40 delivered | Ratio, | |
|---|---|---|---|---|---|---|
| | | | cell | cell | t = 0/initial | Ratio, |
| | Delivery | Initial cell | concentration | concentration | (% of | t = 40/t = 0 |
| Solution # | solution | concentration | (average) | (average) | int) | (% of t = 0) |
| 1 | HBSS, no | 3.95 M | 4.13 M | 1.56 M | 104% | 38% |
| | mix | | | | | |
| 2 | HBSS, mix | 3.95 M | 3.95 M | 4.00 M | 100% | 101% |
| 3 | ISOVUE ®, | 4.47 M | 4.15 M | 3.89 M | 93% | 94% |
| | no mix | | | | | |

The results of Example IV clearly demonstrate that use of a cell settling prevention media (in this case, a dilute solution of ISOVUE® 370 media) allows for delivery of the initial concentration of myoblasts, even after 40 minutes without mixing have elapsed. The myoblast concentration after delivery from the settling prevention media is essentially unchanged after 40 minutes (94% of t=0 concentration), whereas significant numbers of myoblasts are lost after delivery following 40 minutes in HBSS without mixing (only 38% of the t=0 concentration was delivered). These results clearly show the effectiveness of cell settling prevention media for retaining myoblast concentrations in catheter delivery, even without mixing.

### Example V

Human dermal fibroblasts were harvested, counted, and equally divided into two separate tubes. The number of cells in each tube was approximately 375 million cells. The makeup of each tube was as follows:
Solution −: Hanks balanced salt solution with cells
Solution +: Hanks balanced salt solution with cells mixed with ISOVUE®370, adjusted for a specific gravity of 1.060 and an osmolality of 300 mOsm/kg.

The tubes were left at room temperature and pictures were taken at times 0, 40 minutes, and 3 hours (FIG. 5). FIG. 5 illustrates the effect that specific gravity matched solutions with ISOVUE® 370 have, compared to normal Hanks balanced salt solutions on cell settling.

### Example VI

Several tests were made to evaluate the performance of cell delivery fluids across different catheter systems varying the materials, lengths, diameters, and delivery pressures (see also general catheter assemblies below) for different cell types (e.g., fibroblasts and myoblasts—see also cell preparation below) (FIG. 6). Based on the various delivery parameters, e.g., shear rate, shear stress, shear time (see also fluid flow parameters below), percent of live cells resulting from delivery was measured.

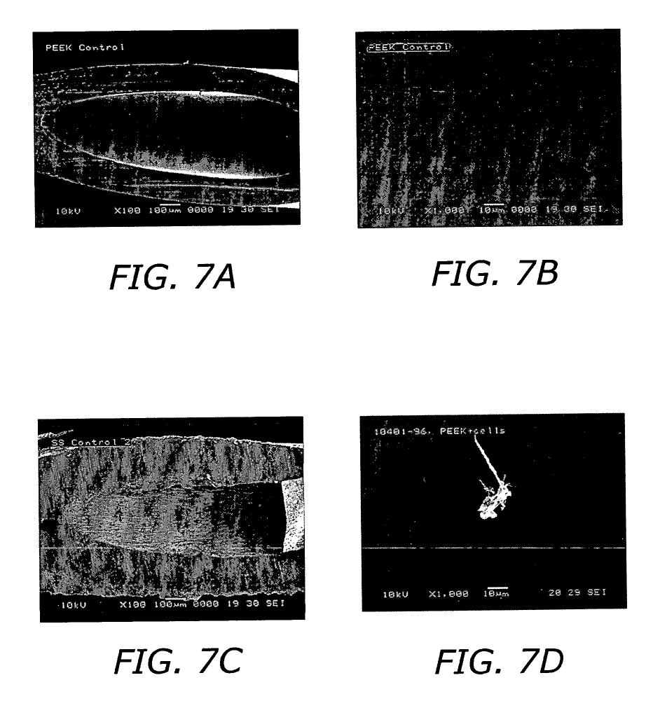

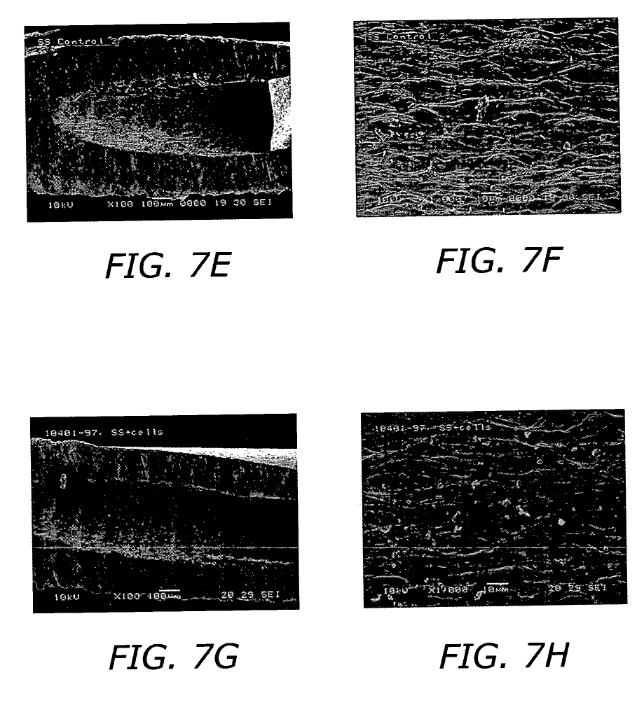

FIGS. 7A through 7H are SEM photographs of lumenal catheter surfaces. In each pair of figures, the image on the left was taken at 100× magnification, and the image on the right at 1000×. The 1000× images are representative areas from the approximate centers of the analogous 100× images: FIGS. 7A and 7B are photographs of PEEK catheter lumens after no exposure to cells; FIGS. 7C and 7D are photographs of PEEK catheter lumens after delivery of myoblasts; FIGS. 7E and 7F are photographs of stainless steel catheter lumens after no exposure to cells; FIGS. 7G and 7H are photographs of stainless steel catheter lumens after delivery of myoblasts. These images indicate that the cell delivery carriers left very few residual cells on the internal surfaces of the catheter.

### General Cell Preparation

Human dermal fibroblasts, (Clonetics, Inc.) or, in later experiments, canine skeletal myoblasts, were cultured in tissue culture flasks using specialty growth media (Clonetics, Inc.). The media was replaced every three days and when confluent, the cells were passaged to propagate the cultures. After it was determined that sufficient cells were available, the cells were rinsed once with HBSS and then dissociated with a 5 min enzymatic wash (0.25% trypsin) at 37° C. The resulting cell suspension was neutralized with serum containing growth media and then centrifuged (800 g) for 10 min to pellet the cells. The supernatant was discarded and the pellet was resuspended in HBSS solution. At this point, the approximate cell concentration was determined by a hemocytometer cell count. The volume of HBSS was adjusted to obtain the desired cell concentration. The initial cell concentration, was calculated from the hemocytometer cell count and HBSS dilution. The final cell suspension was stored under ice for the duration of the experiment.

### General Test Catheter Assembly

The test catheter assemblies were made by bonding segments of PEEK (polyetherether ketone), Pi (polyimide), [or in later experiments, stainless steel (SS)] of various lengths and diameters to LUER-LOCK® stub adaptors with Loctite 401 adhesive after priming with Loctite 7701.

### General Fluid Flow Set-Up

The fluid flow setup consisted of a fluid dispenser (EFD, Model 1500XL) driven by compressed air (max 85 psi) fitted with a 3 cc syringe. The fluid to be dispensed (either the cell suspension of interest or DI water) was loaded into the syringe. The syringe tip was fitted with the test catheter assembly described in the previous section. Delivery time (to the nearest 0.1 second) and pressure (up to 80 psi) can be fixed with this system. To ensure that a suitable volume of cell suspension was delivered, preliminary flow rate measurements were done with DI water.

It is recognized that any of various media may be suitable for the carrier medium, providing that it has the basic characteristics of density matching of intended cells for delivery, a known biologic compatibility, is preferably inexpensive, and is preferably suited for ionic salt solution-type of uses. The delivery device for a preferred cell delivery fluid may utilize components and techniques having a known look and feel to the physician and have ease of functionality due to the characteristics of the medium being that of a fluid with a low viscosity. The density matching technique identified by Applicants permits a more simplified structure of delivery system by obviating the need for complex mixing zones, leuers, or the like. With the cells evenly distributed in all the vessels and tubing of the delivery system, an accurate and consistent number of cells will be delivered- achieving a more consistent therapeutic result. This is achievable without requiring mixing or vibrating the fluid after placement into the delivery catheter. In one embodiment, a catheter is a disposable medical device, although this is not required. A delivery system catheter may be constructed from medical grade plastics and/or other materials, such as stainless steel or other suitable material.

### Example VII

### Purpose

The experiment described in this document was conducted to determine whether the addition of varying concentrations of albumin (0.1%, 0.5%, 1%) to specific gravity matched carrier solutions would adversely affect cell dispersion. The albumin concentrations chosen were based on those reported in the literature.

### Materials

SA=human serum albumin, Fraction V (Sigma # A-1653)
HBBS=Hanks Balanced Salt Solution (Sigma # H-8264)
Isovue 370 (Bracco Diagnostics)
Iso=isotonic ISOVUE® (16.2 ml of ISOVUE® 370+23.8 ml of DI water)
Human skeletal myoblasts
Transparent plastic cuvettes

### Methods

1. The various stock solutions were prepared as such:
a. Solution 1: HBSS
b. Solution 2: HBSS/Iso=67% HBSS: 33% isotonic isovue mixture
c. Solution 3: HBSS+5% SA=50 mg of SA per 1 ml of Solution 1
d. Solution 4: HBSS/Iso+5% SA=50 mg of SA per 1 ml of Solution 2

2. The diluted albumin test solutions were generated from the stock solutions as outlined in Table 6.

TABLE 6

| | | | | Solu- | |
|---|---|---|---|---|---|
| | | | | tion- | |
| | Condition | Solution-1 | Solution-2 | 3 | Solution-4 |
| A | HBSS | 1000 μl | — | — | — |
| B | HBSS/Iso | — | 1000 μl | — | — |
| C | HBSS + 1% SA | 800 μl | — | 200 μl | — |
| D | HBSS + 0.5% SA | 900 μl | — | 100 μl | — |
| E | HBSS + 0.1% SA | 980 μl | — | 20 μl | — |
| F | HBSS/Iso + 1% SA | — | 800 μl | — | 200 μl |
| G | HBSS/Iso + 0.5% SA | — | 900 μl | — | 100 μl |
| H | HBSS/Iso + 0.1% SA | — | 980 μl | — | 20 μl |

3. Human skeletal myoblasts were harvested, counted, and equally divided into the eight tubes containing the different test conditions (A to H). The approximate cell concentration per tube was determined to be 2.15 million cells per ml.

4. A volume of 0.8 ml of well-dispersed cell solution from each tube was transferred to a separate cuvette for evaluation in a UV-VIS spectrophotometer.

5. The cuvettes were read periodically in the spectrophotometer for changes in UV absorption at the following time points: 0, 15, 30, 45, 60 and 90 minutes.

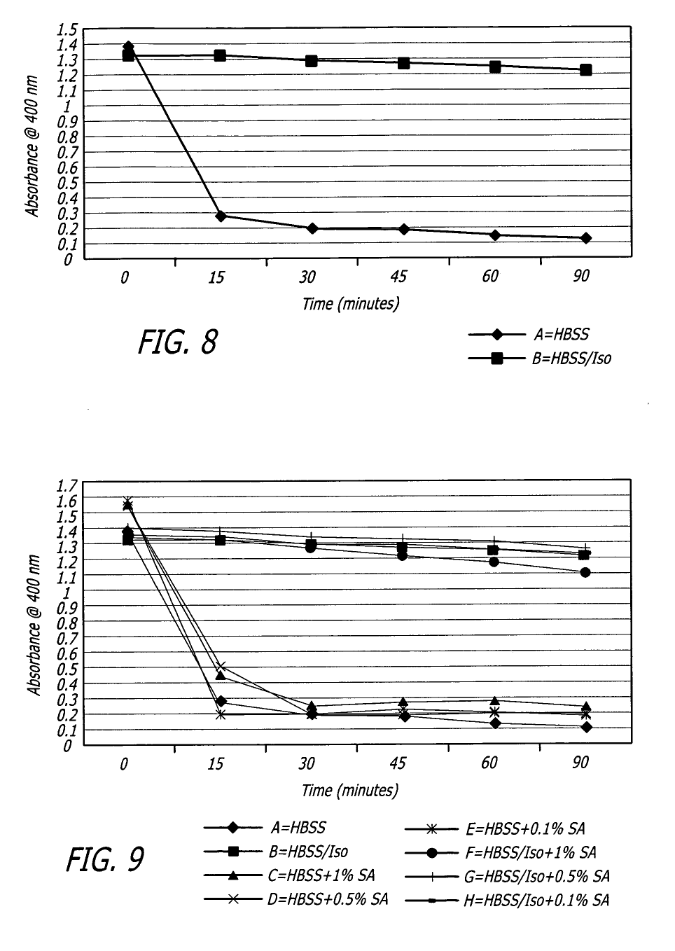

Consistent with our previous findings, the HBSS/Iso solution, as seen in FIG. 8, was able to maintain a dispersed cell solution for the test duration of 90 minutes. This contrasted starkly with the HBSS only condition, in which a significant sedimentation of the cells was observed as early as 15 minutes.

The addition of varying concentrations of albumin to the HBSS/Iso solutions did not appear to interfere with the solutions anti-cell settling property, as seen in FIG. 9. On the other hand, solutions containing albumin, but no isovue, were not able to prevent settling.

This experiment clearly showed that within the range tested (0.1 to 1%), albumin, when added to the HBSS/Iso solution, did not adversely affect cell dispersion.

Another embodiment of this invention includes a method of increasing the efficacy of cell delivery to a patient comprising the steps of providing a carrier liquid for delivering accurate concentrations of cells into a patient. The carrier liquid should have a density which substantially matches the density of the cells to be delivered into the patient when mixed in solution. Also, the solution should have a pH in the range of about 6.0 to 8.0, and more preferably 6.8 to 7.6, and most preferably about 7.0, about 7.2, and about 7.4. Preferably the balanced medium is substantially isotonic as previously described. A further step involves combining the cell delivery of density matched solution of carrier liquid and cells with another medical procedure. The medical procedure may be of various types, and for virtually any medical application. Although the combination may occur at the same time, the combination might not be accomplished simultaneously but rather in a synergistic manner to increase the efficacy of one or both of the cell delivery and the other medical procedure. In one embodiment the medical procedure may be selected from the list of cardiac pacing, cardiac stimulation, cardiac electrical therapy, cardiac pharmacologic therapy, cardiac monitoring, cardiac imaging, cardiac sensing, cardiac mapping, interventional procedures, surgical procedures, infusion procedures, diagnostic procedures, and therapeutic procedures. In another embodiment the medical procedure may be selected from the list of neurologic stimulation, neurologic electrical therapy, neurologic pharmacologic therapy, neurologic monitoring, neurologic imaging, neurologic sensing, neurologic mapping, interventional procedures, surgical procedures, infusion procedures, diagnostic procedures, and therapeutic procedures.

Thus, embodiments of a cell delivery fluid for prevention of cell settling in delivery systems are disclosed. One skilled in the art will appreciate that the present invention can be practiced with embodiments other than those disclosed. The disclosed embodiments are presented for purposes of illustration and not limitation, and the present invention is limited only by the claims which follow.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A cell delivery carrier liquid suspension for delivery of cells into tissue of a mammal, comprising:
a. a volume of cells for delivery to a destination site in a mammal to generate new tissue growth;
b. a carrier liquid having a density which substantially matches the density of the cells when mixed in solution, and the solution having a pH in the range of about 6.0 to 8.0, and being substantially isotonic;
c. a molecular component;
d. wherein the cells remain substantially dispersed in the carrier liquid solution to provide a consistent delivery concentration of cells to the destination site and wherein the molecular component does not substantially alter the viscosity of the cell medium carrier.

2. The cell delivery carrier liquid suspension of claim 1, wherein the molecular component is selected from the group consisting of proteins, non-proteins, and combinations of proteins and non-proteins.

3. The cell delivery carrier liquid suspension of claim 2, wherein the protein comprises human serum albumin (HSA).

4. The cell delivery carrier liquid suspension of claim 2, wherein the proteins comprise growth factors.

5. The cell delivery carrier liquid suspension of claim 4, wherein the growth factors comprise platelet derived growth factors (PDGF).

6. The cell delivery carrier liquid suspension of claim 2, wherein the proteins include extracellular matrices.

7. The cell delivery carrier liquid suspension of claim 6, wherein extracellular matrices comprise structural matrices.

8. The cell delivery carrier liquid suspension claim 7, wherein structural matrices comprise collagens.

9. The cell delivery carrier liquid suspension claim 6, wherein extracellular matrices comprise adhesion matrices.

10. The cell delivery carrier liquid suspension claim 6, wherein extracellular matrices comprise migration matrices.

11. The cell delivery carrier liquid suspension claim 10, wherein the migration matrices are selected from the group consisting of fibronectin, laminin, tenascin, entactin, fibrinogen and fibrin.

12. The cell delivery carrier liquid suspension of claim 2, wherein the non-proteins comprise polysaccharides.

13. The cell delivery carrier liquid suspension claim 2, wherein the non-proteins comprise prostaglandins.

14. A cell delivery liquid suspension for delivery of cells into tissue of a mammal, comprising:
a. a volume of cells for delivery to a destination site in a mammal to generate new tissue growth;
b. a cell density matched solution comprising image enhancing agents having a density which substantially matches the density of the cells when mixed in solution, and the solution having a pH in the range of about 6.0 to 8.0, and being substantially isotonic;
c. a molecular component;
d. wherein the cells remain substantially dispersed in the cell density matched solution to provide a consistent delivery concentration of cells to the destination site and wherein the molecular component does not substantially alter the viscosity of the cell medium carrier.

15. The cell delivery liquid suspension of claim 14, wherein the molecular component is selected from the group consisting of proteins, non-proteins, and combinations of proteins and non-proteins.

16. The cell delivery liquid suspension of claim 15, wherein the image enhancing agents comprise iodine based solutions.

17. The cell delivery liquid suspension of claim 15, wherein the image enhancing agents are labeled to aid in detection.

18. The cell delivery liquid suspension of claim 17, wherein the image enhancing agents comprise iopamidol.

19. A method of preparing a cell-carrier liquid suspension for implantation into a mammal, comprising:
a. selecting a type of cell for implantation into a mammal and identifying the specific gravity of the cell type;
b. selecting a carrier liquid having a specific gravity within a range of specific gravities designed to approximately match the specific gravity of the selected cell type to produce an acceptable delivery ratio of cells at a delivery destination in the mammal;
c. ensuring that the identified liquid has an appropriate osmolality and pH to ensure an acceptable viability of the cells at the delivery destination;
d. selecting a molecular component; and
e. mixing the carrier liquid, the molecular component, and the cells into liquid suspension.

20. The method of claim 19, wherein the molecular component is selected from the group consisting of proteins, non-proteins, and combinations of proteins and non-proteins.
